SEQUENCE LISTING (15 sequences)

SEQ ID NO: 1 - protein, 387 aa, synthetic construct
MALIWATLVVIIIVYAFYELLNNIQKRKSYPADPKGLPIIGHLHLLGKKPHQDFLKLAKTHGPLMYVRLGLVPTIIVSSADTAEKVLKTYDHIFASRPHNEAAYYLAYGQRNMISAKHGPYWRNMRKLCTQHLFSNQKINSFQSMRRQQVEVMIKSLKNGTCDHRVAVYLSAKISSLNADLTCLMVFGKKYMDEDLDKRGFKALVKEVDELAATPNLGDFIPFLGVIDLQGLTRRLKDLSKVFDDFLEKIIDEHVQSGDQKQSKDFVDTMLDIMQSGEAEFQFGRSHIKAILFDMLVAAMGTSATAIEWILTELLQHPHVMKKLQKELEQVVGLEKMVKESDLENLNYLDMVVKERLRLHAVAPLAPHEAMEDCVVNSFHIQKGSGS

SEQ ID NO: 2 - DNA, 1164 bp, synthetic construct
  source 1..1164: mol_type = other DNA
atggctttgatatgggcaacacttgttgttattattattgtgtatgcattttatgagctgctaaacaacatccagaagaggaaaagttatcctgcagatccaaaaggccttcccattataggacatcttcacttgttaggaaaaaagccacaccaagatttcctaaaactagcaaaaacacatggtccacttatgtatgttagactgggactagtacctacaatcattgtctcgtctgcggacacagcggagaaggtcctcaagacatatgatcatatctttgctagtagacctcacaatgaggcagcttattatttagcatatggacagaggaatatgatatccgcaaagcatggaccatactggcgcaacatgcgcaaattgtgcactcagcaccttttcagtaaccaaaagatcaattcatttcaatctatgagaaggcaacaagttgaggttatgatcaaatcacttaaaaatggaacttgtgatcatcgcgttgctgtttatcttagtgcaaagatttcgtctttgaatgctgacttgacttgcttgatggtgtttggaaagaagtacatggatgaagatttggacaagaggggattcaaagctcttgtcaaagaggttgacgaattagcagcaacaccaaatcttggagattttatccccttcctcggtgtaattgatctccagggactcactcgccggctcaaggatctttcaaaagtgtttgatgactttcttgagaagattatcgatgaacatgtccagtctggtgaccagaagcaatctaaagactttgttgacaccatgttggacattatgcaatcaggagaagcagaatttcaatttggccgtagccatataaaagctatactctttgatatgttagttgcagcaatgggcacttcagcaacagcaatagaatggatattaacagagcttcttcagcatcctcatgtgatgaagaaactccaaaaagagttggaacaagtggtaggacttgaaaaaatggtaaaagaatcagacttggagaacttgaactacttagacatggttgtaaaggagcgcttgaggctgcatgccgtagcgccactagcacctcacgaggcaatggaagattgtgtagtcaatagcttccacatacaaaaaggatccggatcctga

SEQ ID NO: 3 - DNA, 3481 bp, synthetic construct
  source 1..3481: mol_type = other DNA
gttttccgaaaacaaaagttcacattacccacttttctctctctaaatttttcaccaaattctatcattatctttctctttcacgatttattccattatttcatattctcaagtaacttaaaccttgtctttttaaaatcaaattcctcgatttttccaagggatctcataaatcatgtaaaagtttttgttacctttgttgaagtgaactcagttgatttgtgattgtttccattagggaagaaaattttgaaataatcgattgggatatcgaagtgaattcaactgatttcgttgaactatgagattgtcggtgaaatacaaaaacaatcaggtattcattaacggttgattctttaatttttttggaattgatacgaaaatggtagttgaaaaaagtgatttagaaatggatctagatcgtattttgtactgttggaccttcacatgaagatctttataagttactatccaaatattggatttaaaggttagacagtcactaaggtattcgatttgggagtttcaggtcaatatttcatccaaggcactatgttggacaacatcaataattatagttatcttttcaatgaattgaagtatttatgtatctttttcgcatgcagaaggacccaaaggagatgaaagtacagaatcaccagccatattttaaagtaaaaaaatatacaaatttttaatttgtaatatgtttgggatacaaatagctagtgaatgattgaaatattttaattttttttaggtcgtatacttggaatagaaaataattttgaaatctatttagtaggtgttttataaattagacattgaatataatatatttttaaaatacataacagataagaaatacaatttttttttaaatacaaattttgattgctaccatgattgtaagtttacatgtgtttgatgactaaaatacaaattttatcttacattttttataaatatacaaacagatgttgtatcatttctgaatgaaaatgaatttacacaagtatatggatacaaatatgcttcttaaataactacaaatccatttggtatacatattggaatcaatacaaagtagtaaagaaattcaaatgtcattatataaacccaaatatgaaaatcataatgaacacaaatacacatatcatacatcttgaaatgaatacaatcaaaagtaaattttaggaataacaaatataaaaatcatattagctctctatagctacagttttgctatttgcactccatataatgcataactatcttgacagtactagaattagtaatacttgtactactttctaaaccaacttgcagccagtcaaataattcaagtagtttctaaaaagccttatagcaacttattcccaaggcttgctttgctatcagacacacaacttgaaatatggctcatttagttgtcttctgacttttttctgaaagcaccatatcttccattaccttgataatcaatacttctcatccacatttttcaaaaataatatctaacattaccaaacacagccattatatgattgagatctctagacccattcaattagagatcttttagaagctaataaagttagtaagtattaggcatggctttgatatgggcaacacttgttgttattattattgtgtatgcattttatgagctgctaaacaacatccagaagaggaaaagttatcctgcagatccaaaaggccttcccattataggacatcttcacttgttaggaaaaaagccacaccaagatttcctaaaactagcaaaaacacatggtccacttatgtatgttagactgggactagtacctacaatcattgtctcgtctgcggacacagcggagaaggtcctcaagacatatgatcatatctttgctagtagacctcacaatgaggcagcttattatttagcatatggacagaggaatatgatatccgcaaagcatggaccatactggcgcaacatgcgcaaattgtgcactcagcaccttttcagtaaccaaaagatcaattcatttcaatctatgagaaggcaacaagttgaggttatgatcaaatcacttaaaaatggaacttgtgatcatcgcgttgctgtttatcttagtgcaaagatttcgtctttgaatgctgacttgacttgcttgatggtgtttggaaagaagtacatggatgaagatttggacaagaggggattcaaagctcttgtcaaagaggttgacgaattagcagcaacaccaaatcttggagattttatccccttcctcggtgtaattgatctccagggactcactcgccggctcaaggatctttcaaaagtgtttgatgactttcttgagaagattatcgatgaacatgtccagtctggtgaccagaagcaatctaaagactttgttgacaccatgttggacattatgcaatcaggagaagcagaatttcaatttggccgtagccatataaaagctatactctttgtacgttattttgatcatcccatctatgttcttctattattaggtttgtttgtgctaatttcccctttctataacaggatatgttagttgcagcaatgggcacttcagcaacagcaatagaatggatattaacagagcttcttcagcatcctcatgtgatgaagaaactccaaaaagagttggaacaagtggtaggacttgaaaaaatggtaaaagaatcagacttggagaacttgaactacttagacatggttgtaaaggagcgcttgaggctgcatgccgtagcgccactagcacctcacgaggcaatggaagattgtgtagtcaatagcttccacatacaaaaaggatccggatcctgattaacttttatgctgttcaaagggatccaaatatttggcctgaacctgaaaagtttttggccgagaggttttctgggagcagtatagacattcgtggacatgacttccaacttttaccatttggctcgggtagaagaagttgtcctgcaatgcagttggcaattgtccttgtccaatttacagtggcacaactggtgcattgctttgattgggagcttccaaatggtatgcagccttgtgaaatggatgttgaggagcactttgggttagcaacaagcagagcaaatcatttaatggccattcctacttatagactaaacaagaatgcttaatctcaaacgatgcaatatggtgcgataatcagttaaaggaagatcaaatgagagattatatacataataataatggagttggctgaaatagttgaccttaattaaaccatttcttctcttctttactggtcatgctatctctcagactaggctcctttgtaatttgtttttctctgtagtatttgtggagctaataagaaatgtattcgtttcacaatgtaatacaaaatacgttgcgaaaatatcataggctaactagcacacaatcataaataaagcaaaaagagaagaaaat

SEQ ID NO: 4 - protein, 306 aa, synthetic construct
TAEKVLKTYDHIFASRPHNEAAYYLAYGQRNMISAKHGPYWRNMRKLCTQHLFSNQKINSFQSMRRQQVEVMIKSLKNGTCDHRVAVYLSAKISSLNADLTCLMVFGKKYMDEDLDKRGFKALVKEVDELAATPNLGDFIPFLGVIDLQGLTRRLKDLSKVFDDFLEKIIDEHVQSGDQKQSKDFVDTMLDIMQSGEAEFQFGRSHIKAILFDMLVAAMGTSATAIEWILTELLQHPHVMKKLQKELEQVVGLEKMVKESDLENLNYLDMVVKERLRLHAVAPLAPHEAMEDCVVNSFHIQKGSGS

SEQ ID NO: 5 - DNA, 3239 bp, synthetic construct
  source 1..3239: mol_type = other DNA
gttttccgaaaacaaaagttcacattacccacttttctctctctaaatttttcaccaaattctatcattatcttttctctttcacgatttattccattatttcatattctcaagtaacttaaaccttgtctttttaaaatcaaattcctcgatttttccaagggatctcataaatcatgtaaaagtttttgttacctttgttgaagtgaactcagttgatttgtgattgtttccattagggaagaaaattttgaaataatcgattgggatatcgaagtgaattcaactgatttcgttgaactatgagattgtcggtgaaatacaaaaacaatcaggtattcattaacggttgattctttaatttttttggaattgatacgaaaatggtagttgaaaaaagtgatttagaaatggatctagatcgtattttgtactgttggaccttcacatgaagatctttataagttactatccaaatattggatttaaaggttagacagtcactaaggtattcgatttgggagtttcaggtcaatatttcatccaaggcactatgttggacaacatcaataattatagttatcttttcaatgaattgaagtatttatgtatctttttcgcatgcagaaggacccaaaggagatgaaagtacagaatcaccagccatattttaaagtaaaaaatatacaaatttttaatttgtaatatgtttgggatacaaatagctagtgaatgattgaaatattttaatttttttttaggtcgtatacttggaatagaaaataattttgaaatctatttagtaggtgttttataaattagacattgaatataatatatttttaaaatacataacagataagaaatacaatttttttttaaatacaaattttgattgctaccatgattgtaagtttacatgtgtttgatgactaaaatacaaattttatcttacattttttataaatatacaaacagatgttgtatcatttctgaatgaaaatgaatttacacaagtatatggatacaaatatgcttcttaaataactacaaatccatttggtatacatattagaatcaatacaaagtagtaaagaaattcaaatgtcattatataaaccctaatatgaaaatcataatgaacacaaatacacatatcatacatcttgaaatgaatacaatcaaaagtaaattttaggaataacaaatataaaaatcatattagctctctatagctacagttttgctatttgcactccatataatgcataactatcttgacagtactagaattagtaatacttgtacaactttctaaaccaacttgcagccagtcaaataattcaagtagtttctaaaaagccttatagcaacttattcccaaggcttgctttgctatcagacacacaacttgaaatatggctcatttagttgtcttctgacttttttctgaaagcaccatatcttccattaccttgataatcaatacttctcatccacatttttcaaaaataatatctaacattaccaaacacagccattatatgattgagatctctagacccattcaattagagatcttttagaagctaataaatttagtaagtattaggcacagcggagaaggtcctcaagacatatgatcatatctttgctagtagacctcacaatgaggcagcttattatttagcatatggacagaggaatatgatatccgcaaagcatggaccatactggcgcaacatgcgcaaattgtgcactcagcaccttttcagtaaccaaaagatcaattcatttcaatctatgagaaggcaacaagttgaggttatgatcaaatcacttaaaaatggaacttgtgatcatcgtgttgctgtttatcttagtgcaaagatttcgtctttgaatgctgacttgacttgcttgatggtgtttggaaagaagtacatggatgaagatttggacaagaggggattcaaagctcttgtcaaagaggttgacgaattagcagcaacaccaaatcttggagattttatccccttcctcggtgtaattgatctccagggactcactcgccggctcaaggatctttcaaaagtgtttgatgactttcttgagaagattatcgatgaacatgtccagtctggtgaccagaagcaatctaaagactttgttgacaccatgttggacattatgcaatcaggagaagcagaatttcaatttggccgtagccatataaaagctatactctttgtacgttattttgatcatcccatctatgttcttctattattaggtttgtttgtgctaatttcccgtttctataacaggatatgttagttgcagcaatgggcacttcagcaacagcaatagaatggatattaacagagtttcttcggcatcctcatgtgatgaagaaactccaaaaagagttggaacaagtggtaggacttgaaaaaatggtaaaagaatcagacttggagaacttgaactacttagacatggttgtaaaggagcgcttgaggctgcatgccgtagcgccactagcacctcacgaggcaatggaagattgtgtagtcaatagcttccacatacaaaaaggatccggatcctgattaacttttatgctgttcaaagggatccaaatatttggcctgaacctgaaaagtttttggccgagaggttttctgggagcagtatagacattcgtggacatgacttccaacttttaccatttggctcgggtagaagaagttgtcctgcaatgcagttggcaattgtccttgtccaatttacagtggcacaactggtgcattgctttgattgggagcttctaaatggtatgcagccttgtgaaatggatgttgaggagcactttgggttagcaacaagcagagcaaatcatttaatagccattcctacttatagactaaacaagaatgcttaatctcaaacgatgcaatatggtgcgataatcaattaaaggaagatcaaatgagagattatatacataataataatggagttggctgaaatagttgaccttaattaaaccatttcttctcttctttactggtcatgctatctctcagactaggctcctttgtaatttgtttttctctgtagtatttgtggagctaataagaaatgtattcgtttcacaatgtaatacaaaatacgttgcgaaaatatcataggctaactagcacacaatcataaataaagcaaaaagagaagaaaat

SEQ ID NO: 6 - DNA, 22 bp, synthetic construct
  source 1..22: mol_type = other DNA
cttgctttgctatcagacacac

SEQ ID NO: 7 - DNA, 20 bp, synthetic construct
  source 1..20: mol_type = other DNA
gcaagtcaagtcagcattca

SEQ ID NO: 8 - DNA, 20 bp, synthetic construct
  source 1..20: mol_type = other DNA
gtgatcatcgcgttgctgtt

SEQ ID NO: 9 - DNA, 21 bp, synthetic construct
  source 1..21: mol_type = other DNA
tccaagatttggtgttgctgc

SEQ ID NO: 10 - DNA, 20 bp, synthetic construct
  source 1..20: mol_type = other DNA
ggtgttatggtcggaatggg

SEQ ID NO: 11 - DNA, 21 bp, synthetic construct
  source 1..21: mol_type = other DNA
cagggtgttcttcaggagcaa

SEQ ID NO: 12 - DNA, 20 bp, synthetic construct
  source 1..20: mol_type = other DNA
tagttgcagcaatgggcact

SEQ ID NO: 13 - DNA, 20 bp, synthetic construct
  source 1..20: mol_type = other DNA
ttccattgcctcgtgaggtg

SEQ ID NO: 14 - DNA, 24 bp, synthetic construct
  source 1..24: mol_type = other DNA
gctgctaaacaacatccagaagag

SEQ ID NO: 15 - DNA, 19 bp, synthetic construct
  source 1..19: mol_type = other DNA
tccgcagacgagacaatga

(19) United States
(12) Patent Application Publication
(10) Pub. No.: US 2024/0263188 A1
(43) Pub. Date: Aug. 8, 2024

(54) APPLICATION OF BFNE GENE IN TOMATO PLANT ARCHITECTURE IMPROVEMENT AND BIOLOGICAL YIELD INCREASE

(71) Applicant: INSTITUTE OF HORTICULTURAL CROPS, XINJIANG ACADEMY OF AGRICULTURAL SCIENCES, Urumqi (CN)

(72) Inventors: Ning LI, Urumqi (CN); Qinghui YU, Urumqi (CN); Qiang HE, Urumqi (CN); Baike WANG, Urumqi (CN); Juan WANG, Urumqi (CN); Tao YANG, Urumqi (CN); Patiguli AISIMUTUOLA, Urumqi (CN)

(21) Appl. No.: 18/641,683

(22) Filed: Apr. 22, 2024

Related U.S. Application Data
(63) Continuation of application No. PCT/CN2022/120881, filed on Sep. 23, 2022.

(30) Foreign Application Priority Data
Oct. 22, 2021 (CN) 202111231381.0

(51) Int. Cl. C12N 15/82; C07K 14/415; C12Q 1/6806; C12Q 1/686; C12Q 1/6895

(52) U.S. Cl. CPC C12N 15/8261; C07K 14/415; C12N 15/8201; C12Q 1/6806; C12Q 1/686; C12Q 1/6895; C07K 2319/00; C12Q 2600/13

(57) ABSTRACT

Provided is an application of a BFNE gene in tomato plant architecture improvement and biological yield increase. A CDS sequence of the BFNE gene is shown as SEQ ID No. 2 in a sequence table; a genome sequence is shown as SEQ ID No. 3 in the sequence table; a coded amino acid sequence is shown as SEQ ID No. 1 in the sequence listing. Also provided is an application of a BFNE protein or a relevant biological material thereof in any one of the following 1) to 5): 1) regulating and controlling a tomato plant architecture; 2) regulating and controlling the tomato yield; 3) breeding transgenic tomatoes with changed plant architecture and/or increased yield; 4) identifying or distinguishing wild tomatoes and cultivated tomatoes; and 5) breeding tomatoes.

### CROSS REFERENCE TO RELATED APPLICATIONS

The present application is a Continuation of International Application Number PCT/CN2022/120881 filed Sep. 23, 2022, which claims priority to Chinese Application Number 202111231381.0 filed Oct. 22, 2021, the disclosure of which are hereby incorporated by reference herein in their entireties.

### INCORPORATION BY REFERENCE

The sequence listing provided in the file entitled C6351-124_PUS124006PCT.xml, which is an Extensible Markup Language (XML) file that was created on Apr. 17, 2024, and which comprises 22,872 bytes, is hereby incorporated by reference in its entirety.

### TECHNICAL FIELD

The present invention relates to the technical field of plant gene engineering, and specifically to the application of BFNE gene in tomato plant architecture improvement and biological yield increase.

### BACKGROUND OF THE INVENTION

Agriculture is the basic industry in China, and grain production is a top priority. Food is the basic condition for human survival. With the growing of population, the gradual decline of arable land, food issues, agricultural development issues have become increasingly prominent. Utilizing high-tech breeding for agricultural science and technology research, vigorously cultivating excellent varieties, and improving the yield of crops per unit area is currently the most direct and effective way to increase production. The number of earth's arable land is limited, and with the continuous growth of the world's population, per capita area of arable land is becoming less and less. In the past 50 years, the area of arable land per capita in the world has actually decreased by more than half. The per capita area of arable land in all parts of China is not optimistic, and it is very important to adhere to the 1.8 billion mu of arable land red line. Xinjiang's arable land area is considered large compared with the other regions, however, the area of arable land does not necessarily mean that the yield is limited by a variety of reasons, such as planting techniques, land salinity, crop varieties and other reasons.

Tomato (Solanum lycopersicum) is an important vegetable and economic crop, which is very popular all over the world. China is the first largest producer of fresh consumptive tomatoes and the third largest producer of processed tomatoes, with a pivotal position in the world tomato market. Due to climatic reasons, tomatoes can only be grown one season a year in Xinjiang. The plant architecture and fruit number are both important factors affecting tomato yield, and decipher the genetic basis of plant architecture and yield will undoubtedly provide important genetic resources for molecular breeding.

### SUMMARY OF THE INVENTION

First, the present invention provides a protein designated BFNE, which is a protein shown in a1) or a2) or a3) or a4) below:
a1) a protein having an amino acid sequence shown in SEQ ID No. 1;
a2) a fusion protein obtained by linking a protein tag at the N-terminal and/or C-terminal of the protein shown in SEQ ID No. 1;
a3) a protein having one or more amino acid residues substituted and/or deleted and/or added in the amino acid sequence shown in SEQ ID No. 1 and being relevant to a plant architecture or a yield of a tomato; and
a4) a protein having 90% identity to the amino acid sequence shown in SEQ ID No. 1, deriving from a tomato and being relevant to a plant architecture or a yield of the tomato.

Of the proteins described in a2) above, the tag refers to a polypeptide or protein that is expressed together in fusion with a target protein by using DNA in vitro recombination, so as to facilitate the expression, detection, tracing and/or purification of the target protein. The tag may be a Flag tag, His tag, MBP tag, HA tag, myc tag, GST tag and/or SUMO tag and the like.

Of the protein described in a3) above, the substitution and/or deletion and/or addition of one or more amino acid residues are substitution and/or deletion and/or addition of no more than 10 amino acid residues.

Of the protein described in a4) above, the protein having 90% identity to the amino acid sequence includes an amino acid sequence having 90% or more, or 91% or more, or 92% or more, or 93% or more, or 94% or more, or 95% or more, or 96% or more, or 97% or more, or 98% or more, or 99% or more homology to the amino acid sequence shown in SEQ ID No. 1 of the present invention.

The protein described in a1) or a2) or a3) or a4) above may be obtained by synthesizing artificially or synthesizing the genes coding for them and then expressing the genes biologically.

The present invention further provides a biological material relevant to the BFNE protein, which is any one of the following A1) to A12):
A1) a nucleic acid molecule encoding the protein;
A2) an expression cassette comprising the nucleic acid molecule of A1);
A3) a recombinant vector comprising the nucleic acid molecule of A1);
A4) a recombinant vector comprising the expression cassette of A2);
A5) a recombinant microorganism comprising the nucleic acid molecule of A1);
A6) a recombinant microorganism comprising the expression cassette of A2);
A7) a recombinant microorganism comprising the recombinant vector of A3);
A8) a recombinant microorganism comprising the recombinant vector of A4);
A9) a cell line comprising the nucleic acid molecule of A1) from a genetically modified plant;
A10) a cell line comprising the expression cassette of A2) from a genetically modified plant;
A11) a cell line comprising the recombinant vector of A3) from a genetically modified plant; and
A12) a cell line comprising the recombinant vector of A4) from a genetically modified plant.

In the above biological materials, the nucleic acid molecule of A1) is a gene shown in 1) or 2) or 3) below:
1) a DNA molecule whose coding sequence is that shown in SEQ ID No. 2 or SEQ ID No. 3;
2) a DNA molecule having 75% or more identity to the nucleotide sequence defined in 1) and encoding the BFNE protein;
3) a DNA molecule hybridizing to the nucleotide sequence defined in 1) or 2) under stringent conditions and encoding the protein.

Wherein the nucleic acid molecule may be DNA such as cDNA, genomic DNA, or recombinant DNA; the nucleic acid molecule may also be RNA such as mRNA or hnRNA.

A person of ordinary skill in the art can readily mutate the nucleotide sequence encoding the BFNE protein of the present invention using known methods, such as directed evolution and point mutation. Those nucleotides that have been artificially modified to have 75% or more identity to the nucleotide sequence encoding the BFNE protein, provided that they encode the BFNE protein and have the same function, are nucleotide sequences derived from and equivalent to the sequence of the present invention.

The term “identity” as used herein refers to sequence similarity to a natural nucleic acid sequence. “Identity” includes a nucleotide sequence having 75% or more, or 85% or more, or 90% or more, or 95% or more identity to the nucleotide sequence encoding a protein consisting of the amino acid sequence shown in SEQ ID No. 1 of the present invention. The identity may be evaluated by the naked eye or by computer software. The identity between two or more sequences may be expressed as a percentage (%) using computer software, which may be used to evaluate the identity between related sequences.

The 75% or more identity described above may be an identity of 80%, 85%, 90% or more than 95%.

Of the above biological materials, the stringent conditions are hybridizing and washing the hybridization membrane in a mixed solution of 2×SSC, 0.1% SDS at 68° C., and repeat the above procedures twice, 5 min each time; alternatively, hybridizing and washing the hybridization membrane in a mixed solution of 0.5×SSC, 0.1% SDS at 68° C., and repeat the above procedures twice, 15 min each time; alternatively, hybridizing and washing the hybridization membrane in a mixed solution of 0.1×SSPE (or 0.1×SSC), 0.1% SDS at 65° C.

Of the above biological materials, the expression cassette comprising the nucleic acid molecule encoding the BFNE protein (BFNE gene-expressing cassette) of A2) refers to a DNA capable of expressing a BFNE protein in a host cell, which may comprise not only a promoter for initiating the transcription of BFNE, but may also comprise a terminator for terminating the transcription of BFNE. Further, the expression cassette may also comprise an enhancer sequence. Promoters that may be used in the present invention include, but are not limited to: constitutive promoters; tissue-, organ- and development-specific promoters and inducible promoters. Suitable transcriptional terminators include, but are not limited to: Agrobacterium nopaline synthase terminator (NOS terminator), Cauliflower mosaic virus CaMV 35S terminator, tml terminator, bean rbcS E9 terminator, and nopaline synthase terminator and octopine synthase terminator.

Recombinant vector containing expression cassette for the BFNE gene may be constructed with existing expression vectors. The plant expression vectors include, for example, binary Agrobacterium tumefaciens vectors and vectors that can be used for biolistics, such as pAHC25, pBin438, pCAMBIA1302, pCAMBIA2301, pCAMBIA1301, pCAMBIA1300, pBI121, pCAMBIA1391-Xa or pCAMBIA1391-Xb. The plant expression vectors may further contain the 3′ end untranslated region of the exogenous gene, that is, it contains polyadenylation signals and any other DNA fragments involved in mRNA processing or gene expression. The polyadenylation signal may direct the incorporation of polyadenylate into the 3′ end of the mRNA precursor, e.g., Agrobacterium crown gall tumor-inducing (Ti) plasmid genes (e.g., the nopaline synthase gene Nos), and the untranslated region of the transcripts at the 3′ end of a gene from plant (e.g., the storage protein genes in soybean) all have similar functions. When constructing plant expression vectors using the genes of the present invention, enhancers may also be used, including translational enhancers or transcriptional enhancers, and these enhancer regions may be an ATG start codon or neighboring region start codons, etc., provided that it is necessary to have the same reading frame as that of the coding sequence in order to ensure the correct translation of the entire sequence. The sources of the translation control signals and start codons are wide-ranging and can be natural or synthetic. Translation initiation regions may be derived from transcription initiation regions or structural genes. In order to facilitate the identification and screening of transgenic plant cells or plants, the plant expression vectors used may be processed, e.g., by incorporating genes encoding enzymes or luminescent compounds that is capable of producing color changes (GUS genes, luciferase gene, etc.), marker genes for antibiotics (e.g., nptII gene conferring resistance to kanamycin and related antibiotics, bar gene conferring resistance to the herbicide hosphinothricin, hph gene conferring resistance to the antibiotic hygromycin, and dhfr gene conferring resistance to amethopterin, and EPSPS gene conferring resistance to glyphosate), or marker genes for resistance to chemicals (e.g., herbicide resistance gene), and mannose-6-phosphate isomerase gene providing the ability to metabolize mannose. In view of the safety of the transgenic plants, the transformed plants can be directly screened for adversity without adding any selective marker genes.

Of the biological materials above, the vector may be a plasmid, mucoid, phage or viral vector.

Of the biological materials above, the microorganism may be a yeast, a bacterium, an alga, or a fungus, such as Agrobacterium.

The present invention also provides a new use of the BFNE protein or the biological materials above.

The present invention provides application of the above BFNE protein or the above biological materials in any of the following 1)-5):
1) regulating and controlling a tomato plant architecture;
2) regulating and controlling the tomato yield;
3) breeding transgenic tomatoes with changed plant architecture and/or increased yield;
4) identifying or distinguishing wild tomatoes and cultivated tomatoes; and
5) breeding tomatoes;

Further, the plant architecture of tomato includes tomato branching. And the regulation and control of the tomato plant architecture is the regulation and control of tomato branching.

The tomato yield comprises fruit number and/or fruit weight of tomato. And the regulation and control of tomato yield is the regulation and control of fruit number and/or weight of tomato.

Still further, the regulation and control of the plant architecture of tomato is to increase the tomato branching, and the regulation and control of the tomato yield is to increase the tomato yield. Specifically, the higher the content and/or activity of the BFNE protein or the higher the expression level of the BFNE gene in tomato, the higher the number of branches (number of lateral branches) in tomato, the higher the number of fruits of the tomato, and the higher the weight of fruits of the tomato.

Of the above applications, the tomato breeding is aimed at cultivating tomato varieties with increased number of branches and/or increased yield.

Of the above applications, the tomato is a wild tomato. The wild tomato may specifically be a cultivar of Micro Tom.

The present invention also provides a method for breeding a transgenic tomato with changed plant architecture and/or increased yield.

The method for cultivating a transgenic tomato with changed plant architecture and/or increased yield provided by the present invention comprises the following steps: obtain a transgenic tomato by increasing the content and/or activity of a protein in a recipient tomato; the transgenic tomato having a higher number of branches (number of lateral branches) and/or a higher yield than the recipient tomato.

In the above method, the yield of the transgenic tomato is higher than that of the recipient tomato as reflected by the fact that the number of fruits of the transgenic tomato is higher than that of the recipient tomato and/or the weight of fruits of the transgenic tomato is higher than that of the recipient tomato.

In the above method, the method of increasing the content and/or activity of a protein in a recipient plant is by overexpressing the protein in a recipient tomato.

Further, the method of overexpression is introducing a gene encoding the protein into the recipient plant.

Still further, the gene encoding the protein is shown as SEQ ID No. 2 in the sequence listing.

In specific embodiments of the present invention, the gene encoding the BFNE protein is introduced into a recipient tomato by a pCAMBIA1300-BFNE recombinant expression vector.

In the above method, the recipient tomato is a wild tomato. The wild tomato may specifically be a cultivar of Micro Tom.

Transgenic tomatoes with changed plant architecture and/or increased yield obtained by the above method also fall within the protection scope of the present invention.

The present invention further provides a method for identifying or distinguishing a wild tomato from a cultivated tomato.

The method for identifying or distinguishing a wild tomato from a cultivated tomato provided by the present invention comprises the following steps: detecting whether the tomato to be tested contains the BFNE protein or its coding gene: if the tomato to be tested contains the BFNE protein (or contains the protein shown in SEQ ID No. 1) or its coding gene (or contains the gene shown in SEQ ID No. 2 or SEQ ID No. 3), the tomato to be tested is a wild tomato; if the tomato to be tested does not contain the BFNE protein (or contains the protein shown in SEQ ID No. 4) or its coding gene (or contains the gene shown in SEQ ID No. 5), the tomato to be tested is a cultivated tomato.

Further, the method of detecting whether a tomato to be tested contains the BFNE protein or its coding gene comprises the following steps: extracting the genomic DNA of the tomato to be tested, and performing PCR amplification with the single-stranded DNA shown in SEQ ID No. 6 and the single-stranded DNA shown in SEQ ID No. 7 to obtain an amplification product; followed by electrophoresis to detect the amplification product, if an amplification product of 777 bp in size is detected, then the tomato to be tested is wild tomato; and if an amplification product of 534 bp in size is detected, then the tomato to be tested is cultivated tomato.

Still further, the cultivar of the wild tomato may be any one of the following: Solanum lycopersicoides, Solanum habrochaites, Solanum pennellii, Solanum chilense, Solanum peruvianum, Solanum corneliomulleri, Solanum neorickii, Solanum chmielewskii, Solanum pimpinellifolium, and Solanum galapagense.

The cultivar of the cultivated tomato may be any of the following: Solanum lycopersicum var. cerasiforme, cultivated tomato M82 Solanum lycopersicum, and cultivated tomato Heinz-1706 Solanum lycopersicum.

The beneficial effects of the present invention are as follows:
(1) In the present invention, a new gene, BFNE, is mined by utilizing the tomato pan-genomic data followed by a multi-genome comparison, which is a time-saving and efficient operation and avoids the disadvantages of time-consuming and labor-intensive by the traditional method of QTL gene localization.
(2) In the present invention, BFNE gene is reported for the first time as a polytropic gene that can regulate and control both the tomato plant architecture and biological yield, which is highly original and lays the foundation for the improvement of tomato varieties and the cultivation of ideal tomato varieties.

### BRIEF DESCRIPTION OF DRAWINGS

FIG. 1 shows a schematic diagram of the presence/absence variant (PAV) of a pleiotropic gene BFNE mined from the tomato pan-genome.

FIG. 2 shows the expression level of BFNE gene in various tissues of wild and cultivated tomatoes.

FIGS. 3A and 3B show the identification of tomatoes transfected with the BFNE gene. FIG. 3A is by PCR identification. Gel bands in lane M represent from top to bottom: 2000 bp; 1500 bp; 1000 bp; 750 bp; 500 bp; 250 bp; and 100 bp. Lanes 1-5 represent Solanum galapagense, Solanum neorickii, cultivated tomato M82 Solanum lycopersicum, Micro Tom tomato, and tomato plants transgenic for the BFNE gene respectively. FIG. 3B shows the identification of BFNE gene expression levels.

FIG. 4 shows the comparison of the plant architectures of tomato plants transfected with the BFNE gene and wild plants.

FIG. 5 shows the comparison of the biological yield of tomato plants transfected with the BFNE gene and wild plants.

FIG. 6 shows the comparison of the fruit weights for red fruits (in grams) of tomato plants transfected with the BFNE gene and wild plants.

FIG. 7 shows the amplification result from wild and cultivated tomatoes by using a pair of primers PAV-F/PAV-R. Gel bands in lane M represent from top to bottom: 2000 bp; 1500 bp; 1000 bp; 750 bp; 500 bp; 250 bp; and 100 bp. Lane 1: Solanum lycopersicoides; Lane 2: Solanum habrochaites; Lane 3: Solanum pennellii; Lane 4: Solanum chilense; Lane 5: Solanum peruvianum; Lane 6: Solanum corneliomulleri; Lane 7: Solanum neorickii; Lane 8: Solanum chmielewskii; Lane 9: Solanum pimpinellifolium; Lane 10: Solanum galapagense; Lane 11: Solanum lycopersicum var. cerasiforme; Lane 12: cultivated tomato M82 Solanum lycopersicum; and Lane 13: cultivated tomato Heinz-1706 Solanum lycopersicum.

### DETAILED DESCRIPTION OF THE INVENTION

The following embodiments facilitate a better understanding of the invention, but do not limit the invention. Test methods used in the following embodiments are conventional methods, if not otherwise specified. Test materials used in the following embodiments are, unless otherwise specified, commercially available from a conventional biochemical reagent store. The quantitative tests in the following embodiments were set up for three repetitive experiments, and the results were averaged.

The Solanum lycopersicoides, Solanum habrochaites, Solanum pennellii, Solanum chilense, Solanum peruvianum, Solanum corneliomulleri, Solanum neorickii, Solanum chmielewskii, Solanum pimpinellifolium, and Solanum galapagense used in the following Examples are all recited in the literature “Spooner D M, Peralta I E, Knapp S. Comparison of AFLPs with Other Markers for Phylogeneti: Inference in Wild Tomatoes [Solanum L. Section Lycopersicon (Mill.) Wettst.] [J]. Taxon, 2005, 54(1):43-61.”, these tomatoes are available to the public from Institute of Horticultural Crops, Xinjiang Academy of Agricultural Sciences and the biological materials are only to be used for the purpose of repeating experiments related to the present invention, and are not to be used for other purposes.

The Solanum lycopersicum var. cerasiforme used in the following Examples is recited in the literature “Ranc N, Muños S, Santoni S, et al. A clarified position for Solanum lycopersicum var. cerasiforme in the evolutionary history of tomatoes (solanaceae) [J]. BMC Plant Biology, 2008, 8.”, the tomato is available to the public from Institute of Horticultural Crops, Xinjiang Academy of Agricultural Sciences and the biological material is only to be used for the purpose of repeating experiments related to the present invention, and are not to be used for other purposes.

The cultivated tomato M82 Solanum lycopersicum used in the following Examples is recited in the literature “Carter J D, Pereira A, Veilleux D. An active ac/ds transposon system for activation tagging in tomato cultivar m82 using clonal propagation. [J]. Plant Physiology, 2013, 162(1): 145-156.”, the tomato is available to the public from Institute of Horticultural Crops, Xinjiang Academy of Agricultural Sciences and the biological material is only to be used for the purpose of repeating experiments related to the present invention, and are not to be used for other purposes.

The cultivated tomato Heinz-1706 Solanum lycopersicum used in the following Examples is recited in the literature “Aureliano B, Naama M, Tecle I Y, et al. The Sol Genomics Network (solgenomics. net): growing tomatoes using Perl [J]. Nucleic Acids Research, 2011, 39(Database issue): 1149-55.”, the tomato is available to the public from Institute of Horticultural Crops, Xinjiang Academy of Agricultural Sciences and the biological material is only to be used for the purpose of repeating experiments related to the present invention, and are not to be used for other purposes.

The Micro Tom tomato used in the following Examples is recited in the literature “Chetty V J, Ceballos N, Garcia D, et al. Evaluation of four Agrobacterium tumefaciens strains for the genetic transformation of tomato (Solanum lycopersicum L.) cultivar Micro-Tom [J]. Plant Cell Reports, 2013, 32(2): 239-247.”, the tomato is available to the public from Institute of Horticultural Crops, Xinjiang Academy of Agricultural Sciences and the biological material is only to be used for the purpose of repeating experiments related to the present invention, and are not to be used for other purposes.

The plant expression vector pCAMBIA1300 used in the following Examples is recited in the literature “Das S S, Sanan M N. A direct method for genetically transforming rice seeds modelled with FHVB2, a suppressor of RNAi [J]. Plant Cell Tissue & Organ Culture, 2015, 120(1): 277-289.”, the vector is available to the public from Institute of Horticultural Crops, Xinjiang Academy of Agricultural Sciences and the biological material is only to be used for the purpose of repeating experiments related to the present invention, and are not to be used for other purposes.

### Example 1 Cloning of the BFNE Gene and Analysis of its Expression Levels in Various Tissues of Wild and Cultivated Tomatoes

### I. Acquisition of the BFNE Gene

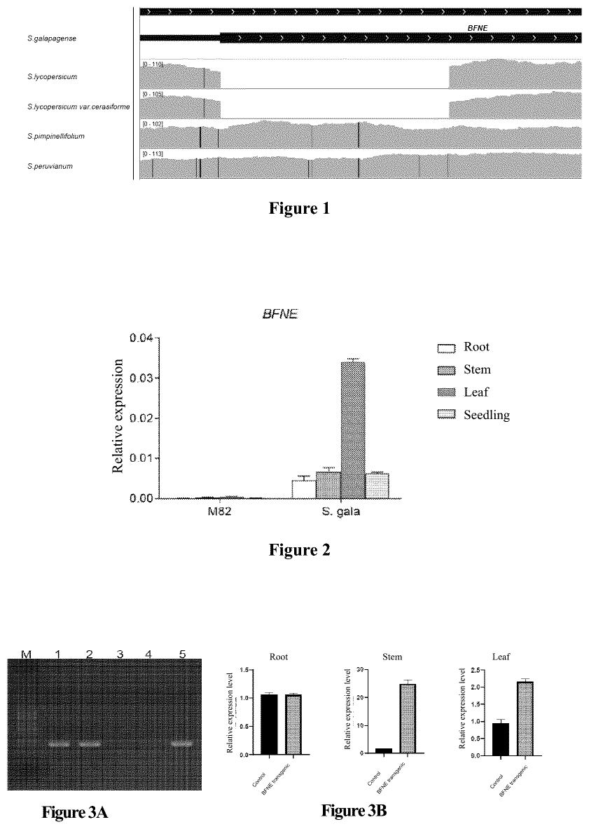

In the present invention, a BFNE gene is mined and cloned utilizing the tomato pan-genomic data by multi-genome comparison, the specific procedures are as follows:
1. High-quality chromosome-scale genome sequences from 11 species of Solanum spp. (9 wild and 2 cultivated tomatoes) were assembled using the PacBio+Hi-C strategy.
2. Four de novo prediction programs including RepeatScout, LTR-FINDER, MITE-hunter and PILER-DF were applied to construct a repetitive sequence database. Repetitive sequences were identified and annotated by homolog searching of the final merged database using RepeatMasker.
3. Analysis of core genes and dispensable genes, including functional enrichment analysis, conservation analysis, repetitive sequence region enrichment analysis and the likes.
4. Genomic SVs detection was performed based on genomic sequence comparison, followed by analysis of SVs features, such as comparison of SVs type, length distribution, genomic distribution, repeats and the likes.
5. The wild tomato-specific gene BFNE was finally found, and the result is shown in FIG. 1.

The sequence for the BFNE gene specific in wild tomato is shown as SEQ ID No. 3 in the sequence listing, the CDS sequence is shown as SEQ ID No. 2 in the sequence listing, and the amino acid sequence of the BFNE protein encoded by the BFNE gene is shown as SEQ ID No. 1 in the sequence listing.

### II. Analysis of BFNE Gene Expression Levels in Various Tissues of Wild and Cultivated Tomatoes

Tissue samples of roots, stems, leaves, and seedlings from wild tomato Solanum galapagense and cultivated tomato M82 were collected, RNA was extracted for reverse transcription, and quantitative RT-PCR was performed using a LightCycler96 real-time PCR system. In the qRT-PCR experiments, Actin gene of tomato was used as an internal control and three samples (biological replicates) were used for each treatment. Primer sequences were specified as follows:

| | RTBF4-F: |
|---|---|
| (SEQ ID No. 8) | |
| | GTGATCATCGCGTTGCTGTT; |
| | RTBF4-R: |
| (SEQ ID No. 9) | |
| | TCCAAGATTTGGTGTTGCTGC; |
| | Actin-F: |
| (SEQ ID No. 10) | |
| | GGTGTTATGGTCGGAATGGG; |
| | Actin-R: |
| (SEQ ID NO. 11) | |
| | CAGGGTGTTCTTCAGGAGCAA. |

The results are shown in FIG. 2. The results showed that the expression levels of BFNE gene were higher in roots, stems, leaves, and seedlings from the wild tomato Solanum galapagense than those from cultivated tomato M82, especially in leaves.

### Example 2. Application of BFNE Gene in Tomato Plant Architecture Improvement and Biological Yield Increase

### I. Preparation of a BFNE Gene Transgenic Tomato

### 1. Construction of a Recombinant Expression Vector

The BFNE gene shown in SEQ ID No. 2 was integrated into the plant expression vector pCAMBIA1300 between the XbaI and KpnI restriction sites by enzyme digestion and ligation, and the other sequence in the plant expression vector pCAMBIA1300 were kept unchanged to obtain the pCAMBIA1300-BFNE recombinant expression vector.

### 2. Acquisition of Transgenic Tomato Plants

The pCAMBIA1300-BFNE recombinant expression vector obtained in step 1 above was introduced into GV3101 Electrocompetent cells (Beijing Biomed Gene Technology Co., Ltd., Item No. BC308-01), and the recombinant bacterium pCAMBIA1300-BFNE/GV3101 was obtained through identification. The recombinant bacterium pCAMBIA1300-BFNE/GV3101 was then transformed into Micro Tom tomato by leaf disk transformation to obtain a BFNE gene transgenic tomato of T₀ generation.

### 3. Identification of the Transgenic Tomato Plants

T₀ generation BFNE gene transgenic tomato plants were planted in the greenhouse and self-crossed to obtain T₁ generation BFNE gene transgenic tomato plants, which were identified.

### 1) Identification by PCR

Genomic DNAs from Solanum galapagense, Solanum neorickii, cultivated tomato M82 Solanum lycopersicum, Micro Tom tomato and T₁ generation of BFNE gene transgenic tomato plants were extracted. respectively by using BFNE-F: GCTGCTAAACAACATCCAGAAGAG(SEQ ID No. 14) and BFNE-R: TCCGCAGACGAGACAATGA(SEQ ID No.15) primers for PCR amplification and the PCR amplification products were detected by electrophoresis.

The results are shown in FIG. 3, panel A. As can be seen from the figure, a band of 186 bp was amplified in Solanum galapagense, Solanum neorickii and BFNE gene transgenic tomato plants, while no bands were amplified in either cultivated tomato M82 Solanum lycopersicum or Micro Tom tomato.

### 2) Detection of the Expression Levels of the BFNE Gene

BFNE gene expression levels were detected by RT-PCR in roots, stems and leaves of T₁ generation tomato plants identified as positive by PCR, Micro Tom tomato was used as a control. Sequences of the primers specific for the BFNE gene were as follows: RTBF1-F: TAGTTGCAGCAATGGGCACT (SEQ ID No. 12); RTBF1-R: TTCCATTGCCTCGTGAGGTG (SEQ ID No. 13). Sequences of the control gene are as follows: Actin-F: primers for the internal GGTGTTATGGTCGGAATGGG (SEQ ID No. 10); Actin-R:

| (SEQ ID NO. 11) | |
|---|---|
| | CAGGGTGTTCTTCAGGAGCAA. |

The results are shown in FIG. 3, panel B. As can be seen from the figure, the expression level of BFNE gene was significantly increased in both stems and leaves of tomato plants transfected with BFNE gene in T₁ generation than that in wild type Micro Tom tomato.

### II. Analysis of Plant Architecture and Yield in BFNE Gene Transgenic Tomato

The T₁ generation of BFNE gene transgenic tomato plants and wild-type plants (Micro Tom tomato) were planted in the greenhouse at the same time, the phenotypes were observed at the ripening stage, and the number of branches and the number and weight of fruits were counted.

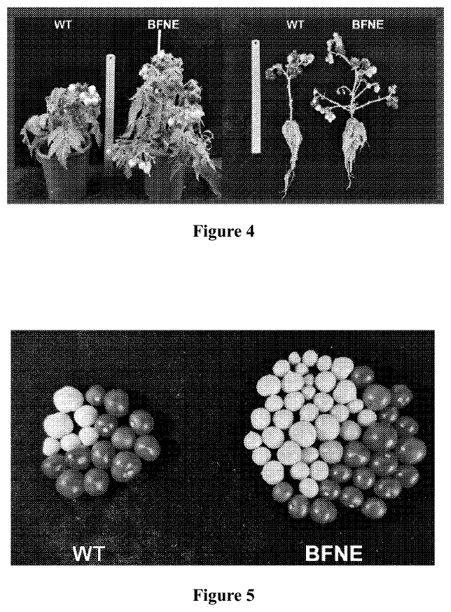

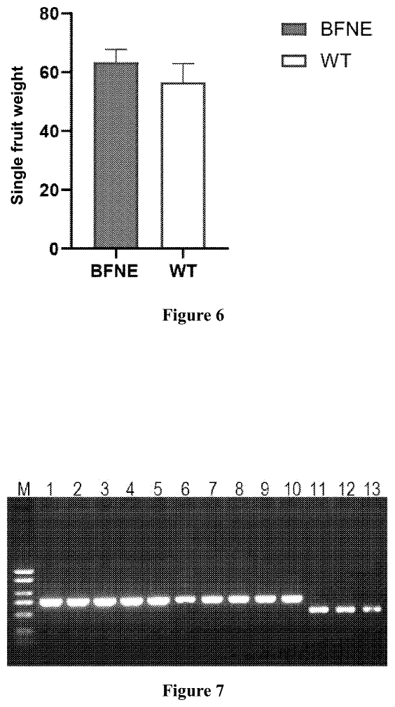

The results showed that there was an increase in the number of branches (lateral branching) in the BFNE gene transgenic tomato, while there was no branching in wild-type plants (Table 1 and FIG. 4). Moreover, the total number of fruits per plant and total fruit weight of the BFNE gene transgenic tomatoes were higher than those of wild-type tomatoes (Table 2, FIGS. 5 and 6).

TABLE 1
Branch numbers of the BFNE gene transgenic
tomatoes and wild-type tomatoes

| | Branch numbers (unit) | | |
|---|---|---|---|
| Name | Replicate 1 | Replicate 2 | Replicate 3 |
| WT | 0 | 0 | 0 |
| BFNE gene | 5 | 6 | 5 |
| transgenic tomatoes | | | |

Note:
Replicates 1, 2, and 3 denote three replicated individual plants selected for WT and BFNE gene transgenic tomato, respectively.

TABLE 2
Number and total weight of fruits of BFNE gene
transgenic tomatoes and wild-type tomatoes

| | | Total | | Total | Total | Total |
|---|---|---|---|---|---|---|
| | Numbers | weight | Numbers | weight | fruit | numbers |
| | of the | of the | of the | of the | weight | of fruit |
| | red | red | green | green | per | per |
| | fruit | fruits | fruit | fruits | plant | plant |
| Name | (unit) | (grams) | (unit) | (grams) | (grams) | (unit) |
| BFNE-1 | 23 | 61.31 | 34 | 49.58 | 110.89 | 57 |
| BFNE-2 | 22 | 60.36 | 30 | 46.56 | 106.92 | 52 |
| BFNE-3 | 26 | 68.42 | 27 | 44.29 | 112.71 | 53 |
| WT-1 | 18 | 53.97 | 5 | 11.79 | 65.76 | 23 |
| WT-2 | 19 | 63.76 | 3 | 2.43 | 66.91 | 22 |
| WT-3 | 18 | 51.97 | 6 | 13.87 | 65.84 | 24 |

Note:
BFNE-1, BFNE-2 and BFNE-3 are all BFNE gene transgenic tomato plants of T₁ generation; WT-1, WT-2 and WT-3 are all wild tomatoes (Micro Tom tomato); 1, 2 and 3 denote three replicated individual plants selected for BFNE gene transgenic tomato plants of T₁ generation and wild tomatoes (Micro Tom tomato), respectively.

The above results showed that the BFNE gene from wild tomato could increase the number of branches and fruit yield of Micro Tom tomato. BFNE has an important application value in molecular breeding for improving tomato plant architecture and increasing tomato biological yield.

### Example 3 Application of the BFNE Gene in Identifying or Differentiating Wild Tomatoes from Cultivated Tomatoes

The sequence of BFNE gene in wild tomato is shown in SEQ ID No. 3 in the sequence listing, and the amino acid sequence of BFNE protein encoded thereby is shown in SEQ ID No. 1 in the sequence listing; the sequence of BFNE gene in the cultivated tomato is shown in SEQ ID No. 5 in the sequence listing, and the amino acid sequence of the BFNE protein encoded thereby is shown in SEQ ID No. 4 in the sequence listing, therefore, it is possible to identify or distinguish wild tomatoes and cultivated tomatoes according to the BFNE protein or the sequence of its coding gene. The specific methods are described below:

### Experimental Materials:

1: Solanum lycopersicoides
2: Solanum habrochaites
3: Solanum pennellii
4: Solanum chilense
5: Solanum peruvianum
6: Solanum corneliomulleri
7: Solanum neorickii
8: Solanum chmielewskii
9: Solanum pimpinellifolium
10: Solanum galapagense
11: Solanum lycopersicum var. cerasiforme
12: Cultivated tomato M82 Solanum lycopersicum
13: Cultivated tomato Heinz-1706 Solanum lycopersicum.

Among them, numbers 1-10 are all wild tomatoes; and numbers 11-13 are all cultivated tomatoes.

Experimental methods: Genomic DNA of tomato to be tested was extracted and amplified using primers PAV-F: CTTGCTTTGCTATCAGACACAC (SEQ ID No. 6) and PAV-R: GCAAGTCAAGTCAGCATTCA (SEQ ID No. 7) to obtain the products, followed by electrophoresis was carried out for detecting the size of the products.

Results: a gel band of 777 bp in size of the amplification product was obtained in all wild tomatoes, and a gel band of 534 bp in size of the amplification product was obtained in all cultivated tomatoes (FIG. 7).

Therefore, in practical applications, the following method may be used to identify whether the tomato species to be tested is wild tomato or cultivated tomato: extracting the genomic DNA of the tomato to be tested, and performing amplification with a pair of primers PAV-F/PAV-R to obtain an amplication product, followed by electrophoresis to detect the amplification product, if an amplification product of 777 bp in size is detected, then the tomato to be tested is wild tomato; and if an amplification product of 534 bp in size is detected, then the tomato to be tested is cultivated tomato.

The foregoing is only preferred embodiments of the present invention, and it should be noted that for a person of ordinary skill in the art, a number of improvements and embellishments may be made without departing from the technical principles of the present invention, and these improvements and embellishments shall also be regarded as the scope of protection of the present invention.

### INDUSTRIAL APPLICATIONS

In the present invention, a wild tomato gene was firstly mined using pan-genome technique, which was named as BFNE (branch and fruit number enhancer) gene, and then the BFNE gene was overexpressed in wild tomato (Micro Tom tomato) to obtain BFNE gene transgenic tomato. It was proved that by experiments that the BFNE gene transgenic tomato showed an increase in the number of branches, and an increase in the number and weight of fruits than Micro Tom tomato. This indicates that the BFNE gene could improve the tomato plant architecture and increase the tomato yield. The present invention also figures out that the BFNE gene may also be used to identify or distinguish wild tomatoes and cultivated tomatoes, and may be used for cross-breeding wild tomatoes and cultivated tomatoes and improving tomato variety.

What is claimed is:

1. A protein, being a protein shown in a1) or a2) or a3) or a4) below:
a1) a protein having an amino acid sequence shown in SEQ ID No. 1;
a2) a fusion protein obtained by linking a protein tag at the N-terminal and/or C-terminal of the protein shown in SEQ ID No. 1;
a3) a protein having one or more amino acid residues substituted and/or deleted and/or added in the amino acid sequence shown in SEQ ID No. 1 and being relevant to a plant architecture or a yield of a tomato; and
a4) a protein having 90% identity to the amino acid sequence shown in SEQ ID No. 1, deriving from a tomato and being relevant to a plant architecture or a yield of the tomato.

2. A biological material relevant to a protein, being any one of the following A1) to A12):
A1) a nucleic acid molecule encoding the protein;
A2) an expression cassette comprising the nucleic acid molecule of A1);
A3) a recombinant vector comprising the nucleic acid molecule of A1);
A4) a recombinant vector comprising the expression cassette of A2);
A5) a recombinant microorganism comprising the nucleic acid molecule of A1);
A6) a recombinant microorganism comprising the expression cassette of A2);
A7) a recombinant microorganism comprising the recombinant vector of A3);
A8) a recombinant microorganism comprising the recombinant vector of A4);
A9) a cell line comprising the nucleic acid molecule of A1) from a genetically modified plant;
A10) a cell line comprising the expression cassette of A2) from a genetically modified plant;
A11) a cell line comprising the recombinant vector of A3) from a genetically modified plant; and
A12) a cell line comprising the recombinant vector of A4) from a genetically modified plant;
wherein the protein is a protein shown in a1) or a2) or a3) or a4) below:
a1) a protein having an amino acid sequence shown in SEQ ID No. 1;
a2) a fusion protein obtained by linking a protein tag at the N-terminal and/or C-terminal of the protein shown in SEQ ID No. 1;
a3) a protein having one or more amino acid residues substituted and/or deleted and/or added in the amino acid sequence shown in SEQ ID No. 1 and being relevant to a plant architecture or a yield of a tomato; and
a4) a protein having 90% identity to the amino acid sequence shown in SEQ ID No. 1, deriving from a tomato and being relevant to a plant architecture or a yield of the tomato.

3. A biological material according to claim 2, wherein the nucleic acid molecule of A1) is a gene shown in 1) or 2) or 3) below:
1) a DNA molecule whose coding sequence is that shown in SEQ ID No. 2 or SEQ ID No. 3;
2) a DNA molecule having 75% or more identity to the nucleotide sequence defined in 1) and encoding the protein; and
3) a DNA molecule hybridizing to the nucleotide sequence defined in 1) or 2) under stringent conditions and encoding the protein.

4. Any of the following methods:
1) a method of regulating and controlling a tomato plant architecture;
2) a method of regulating and controlling the tomato yield;
3) a method of breeding transgenic tomatoes with changed plant architecture and/or increased yield;
4) a method of identifying or distinguishing wild tomatoes and cultivated tomatoes; and
5) a method of breeding tomatoes.

5. The method according to claim 4, wherein 1)-5) all use a protein or a biological material relevant to the protein.

6. The method according to claim 5, wherein the protein is a protein shown in a1) or a2) or a3) or a4) below:
a1) a protein having an amino acid sequence shown in SEQ ID No. 1;
a2) a fusion protein obtained by linking a protein tag at the N-terminal and/or C-terminal of the protein shown in SEQ ID No. 1;
a3) a protein having one or more amino acid residues substituted and/or deleted and/or added in the amino acid sequence shown in SEQ ID No. 1 and being relevant to a plant architecture or a yield of a tomato; and
a4) a protein having 90% identity to the amino acid sequence shown in SEQ ID No. 1, deriving from a tomato and being relevant to a plant architecture or a yield of the tomato.

7. The method according to claim 5, wherein the biological material is any one of the following A1) to A12):
A1) a nucleic acid molecule encoding the protein;
A2) an expression cassette comprising the nucleic acid molecule of A1);
A3) a recombinant vector comprising the nucleic acid molecule of A1);
A4) a recombinant vector comprising the expression cassette of A2);
A5) a recombinant microorganism comprising the nucleic acid molecule of A1);
A6) a recombinant microorganism comprising the expression cassette of A2);
A7) a recombinant microorganism comprising the recombinant vector of A3);
A8) a recombinant microorganism comprising the recombinant vector of A4);
A9) a cell line comprising the nucleic acid molecule of A1) from a genetically modified plant;
A10) a cell line comprising the expression cassette of A2) from a genetically modified plant;
A11) a cell line comprising the recombinant vector of A3) from a genetically modified plant; and
A12) a cell line comprising the recombinant vector of A4) from a genetically modified plant.

8. The method according to claim 4, wherein the regulation and control of the tomato plant architecture is the regulation and control of tomato branching.

9. The method according to claim 4, wherein the regulation and control of tomato yield is the regulation and control of fruit number and/or weight of tomato.

10. The method according to claim 4, wherein the tomato is a wild tomato.

11. The method according to claim 4, wherein the method for breeding a transgenic tomato with changed plant architecture and/or increased yield, comprising the steps of: obtain a transgenic tomato by increasing the content and/or activity of a protein in a recipient tomato; the transgenic tomato having a higher number of branches and/or a higher yield than the recipient tomato.

12. The method according to claim 11, wherein the yield of the transgenic tomato is higher than that of the recipient tomato as reflected by the fact that the number of fruits of the transgenic tomato is higher than that of the recipient tomato and/or the weight of fruits of the transgenic tomato is higher than that of the recipient tomato.

13. The method according to claim 11, wherein the method of increasing the content and/or activity of a protein in a recipient plant is by overexpressing the protein in a recipient tomato.

14. The method according to claim 13, wherein the method of overexpression is introducing a gene encoding the protein into the recipient plant.

15. The method according to claim 14, wherein the gene encoding the protein is shown as SEQ ID No. 2 in the sequence listing.

16. The method according to claim 15, wherein the recipient tomato is a wild tomato.

17. A transgenic tomato prepared by the method claim 11.

18. The method according to claim 4, wherein the method for identifying or distinguishing a wild tomato from a cultivated tomato, comprising the steps of: detecting whether the tomato to be tested contains a protein or a gene encoding thereof: if the tomato to be tested contains the protein or the gene encoding thereof, the tomato to be tested is a wild tomato; if the tomato to be tested does not contain the protein or the gene encoding thereof, the tomato to be tested is a cultivated tomato.

19. The method according to claim 18, wherein the gene encoding the protein is a DNA molecule shown in SEQ ID No. 2 or SEQ ID No. 3.

20. The method according to claim 19, wherein the method of detecting whether a tomato to be tested contains a protein or a gene encoding thereof comprises the following steps: extracting the genomic DNA of the tomato to be tested, and performing PCR amplification with the single-stranded DNA shown in SEQ ID No. 6 and the single-stranded DNA shown in SEQ ID No. 7 to obtain an amplification product; followed by electrophoresis to detect the amplification product, if an amplification product of 777 bp in size is detected, then the tomato to be tested is wild tomato; and if an amplification product of 534 bp in size is detected, then the tomato to be tested is cultivated tomato.
